(19) United States
(12) Patent Application Publication
(10) Pub. No.: US 2005/0080465 A1
(43) Pub. Date: Apr. 14, 2005

(54) Device and method for treatment of external surfaces of a body utilizing a light-emitting container

(76) Inventors: Brian Zelickson, Minneapolis, MN (US); Robert A. Ganz, Minnetonka, MN (US)

Correspondence Address: Schwegman, Lundberg, Woessner & Kluth, P.A., P.O. Box 2938, Minneapolis, MN, 55402, US

(21) Appl. No.: 10/941,043

(22) Filed: Sep. 14, 2004

Related U.S. Application Data
(63) Continuation of application No. PCT/US03/08156, filed on Mar. 17, 2003.
(60) Provisional application No. 60/364,976, filed on Mar. 15, 2002.

(51) Int. Cl. A61F 5/00

(52) U.S. Cl. 607/88

(57) ABSTRACT

A device includes a patch or bandage, applied on or adjacent to a specific external surface of a human or animal body part, which delivers light of varying wavelengths, intensity and duration for treatment of various diseases or abnormalities.

### RELATED APPLICATION

This application is a continuation under 35 U.S.C. 111(a) of International Application No. PCT/US03/08156 filed Mar. 17, 2003 and published in English as WO 03/077996 A2 on Sep. 25, 2003, which claims priority under 35 USC119(e) from U.S. Provisional Application Ser. No. 60/364,976, filed Mar. 15, 2002, which applications and publication are incorporated herein by reference.

### FIELD OF THE INVENTION

This invention relates to the field of medical treatment, and more specifically to a method and apparatus of treatment by light emission.

### BACKGROUND

There are many medical conditions that are and can be treated with light based therapy. These include but are not limited to cutaneous disorders such as acne, psoriasis, eczema, warts, basal and squamous cell cancer, herpes, acne, photodamage, vitiligo, ulcers and superficial infections, as well as dental disorders such as gingivitis and tooth discoloration. The light emitting devices used to treat these disorders range from ultraviolet light boxes, mercury arc lamps, Xenon arc lamps, and a variety of lasers.

It is often difficult and impractical to confine the correct amount of illumination to the tissue that needs it while not exposing the normal tissue. One way of doing this is to couple an optical fiber to the light source and focus the light only on the areas in which need treatment. This is time consuming and not good for odd shaped or convoluted areas.

### SUMMARY

The present system provides a concept of incorporating therapeutic light energy in a container, such as a patch or bandage, that can be affixed to, or held adjacent to or over, external surfaces of a human or animal body, for the treatment of body sites such as skin or teeth. The light can be specified as having certain wavelengths, energy pulse durations, and directed specifically to the area needing treatment. The light-containers can be constructed to contain active topical agents, drug delivery mechanisms, and have the ability to elaborate electrical and thermal energy to enhance the therapeutic effects.

In one example, the present system includes a process for producing a container that emits light energy, that adheres to or can be positioned adjacent to an external surface of the body. The system includes a patch or bandage shape member and a light source of specific wavelength, intensity and duration of exposure. In various embodiments, the light source can include a light source comprised of a cool light device, a light source comprised of a chemiluminescent material, a light source comprised of an electroluminescent material, a light source composed of a light emitting diode, and a light source comprised of a light-emitting polymer. The light source can be totally self contained or have an external power supply. The patch or bandage can be adapted to be affixed, applied to, or positioned adjacent to, the treatment area of skin or teeth, and can include a hydro colloid dressing, a flexible adherent material, a moldable polymer material, or a flexible water repellant material.

One aspect includes a bandage or patch that has a reflecting surface so as to direct the light and reflected light to the treatment surface. One aspect includes a bandage or patch that contains other topical preparations to enhance the effect of the light therapy. One aspect includes a bandage or patch that contains a topical delivery system for driving topical preparations into the treatment area.

The bandage or patch can include an ability to produce an electrical field that can affect the treatment area by driving product into the treatment area or creating electrical or thermal energy that can enhance the therapeutic effect of the light energy. The bandage or patch can include the ability to create a thermal reaction such as a hyper- or hypothermal reaction to enhance the therapeutic effect of the light energy.

One aspect provides a dentifrice that can be custom molded in a specific configuration so as to be used as a dental tray for teeth whitening or cleaning.

One aspect provides a patch or bandage that can be applied either by adhesives, straps, ties or binders of any type.

### BRIEF DESCRIPTION OF THE DRAWINGS

FIG. 1 shows a perspective view of a light-emitting system according to one embodiment.

FIG. 2 shows a perspective view of a light-emitting system according to one embodiment.

FIG. 3 shows a dental tray light-emitting device according to one embodiment.

FIG. 4 shows a pair of dental trays applied according to one embodiment.

FIG. 5 shows a light-emitting patch according to one embodiment.

FIG. 6 shows light-emitting patches applied to one or more facial areas in accordance with one embodiment.

FIG. 7 shows a light emitting patch for removing unwanted hair according to one embodiment.

FIG. 8 shows a front view of a light-emitting face mask according to one embodiment.

FIG. 9 shows a perspective view of the light-emitting face mask of FIG. 8.

FIG. 10 shows a side view of a light-emitting patch according to one embodiment.

FIG. 11 shows a side view of a plurality of light-emitting patches in accordance with one embodiment.

FIG. 12 shows a top view of a light-emitting patch according to one embodiment.

FIG. 13 shows a side view of a light-emitting patch according to one embodiment.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that other embodiments may be utilized and that structural changes may be made without departing from the scope of the present invention. Therefore, the following detailed description is not to be taken in a limiting sense, and the scope of the present invention is defined by the appended claims and their equivalents.

By employing a variety of luminescent materials such as light emitting diodes, chemiluminescence, and efficient energy sources that are being used for glow sticks and flashlights, the present system produces tiny light sources and embeds them in a bandage or patch to conform to the treatment area thus confining the light energy to the area needed to be treated.

Topical patches and bandages are currently being used not only to help heal wounded tissue but for medicinal purposes. By coupling active ingredients into the patch or bandage and by constructing the patch or bandage in order to enhance penetration of such ingredients one constructs a therapeutic device. While there have been patches that employ materials of different polarity in order to create an electrical current there have been no bandages or patches devised to produce light energy in a sufficient wavelength, intensity and duration in order to effectively treat the skin or teeth.

Whereas current therapeutic light sources employ high energy lasers and arc lamps needing to be connected to external power sources, the development of other cool light sources have made it feasible to treat disorders of the skin or teeth, when combined with a way to apply the light source to the treatment area for creating sufficient light energy.

One such cool light source is chemiluminescence. This technique allows the ability to produce light from a chemical reaction. Several chemiluminescence substances luminal and lucigenin were discovered in 1928 and 1935, respectively. These were followed by the development of a series of organic soluble chemiluminescent materials in the early 1960s. These materials as disclosed by Bollyky et al., U.S. Pat. No. 3,597,362 were more efficient than the prior aqueous compounds.

Basically, these chemical reactions consist of two components: an oxilaic ester and a hydrogen peroxide along with an efficient fluorescer and a catalyst may be added to help control the reaction.

Examples of fluorescent compounds include: the conjugated polycyclic aromatic compounds which include anthracene, benzanthracene, phenanthrene, naphthacene, pentacene, perylene, perylene, violanthrone, and the like and their substituted forms. Typical substituents for all of these are phenyl, lower alkyl (C.sub.1 -C.sub.6), chloro, bromo, cyano, alkoxy (C.sub.1 -C.sub.16), and other like substituents, which do not interfere with the light-generating reaction, contemplated herein.

Some fluorescers are 9,10-bis(phenylethynyl) anthracene, 1-methoxy-9,10-bis(phenylethynyl)anthracene, perylene, 1,5-dichloro 9,10-bis(phenylethynyl) anthracene, rubrene, monochloro and dichloro substituted 9,10-bis(phenylethynyl) anthracene, 5,12-bis(phenylethynyl) tetracene, 9,10-diphenyl anthracene, and 16,17-dihexyloxyviolanthrone.

The lifetime and intensity of the chemiluminescent light emitted can be regulated by the use of certain regulators such as:
(1) by the addition of a catalyst which changes the rate of reaction of hydroperoxide. Catalysts which accomplish that objective include those described in M. L. Bender, Chem. Revs., Vol. 60, p. 53 (1960), which is incorporated herein by reference. Also, catalysts which alter the rate of reaction or the rate of chemiluminescence include those accelerators of U.S. Pat. No. 3,775,366, and decelerators of U.S. Pat. Nos. 3,691,085 and 3,704,231, all of which are incorporated herein by reference, or
(2) by the variation of hydrogenperoxide. Both the type and the concentration of hydrogen peroxide are important for the purposes of regulation.

Of the catalysts tried, sodium salicylate and various tetraalkylammonium salicylates have been the most widely used. Lithium carboxylic acid salts, especially lithium salicylate, lithium 5-t-butyl salicylate and lithium 2-chlorobenzoate are excellent catalysts for low temperature hydrogen peroxide/oxalate ester/fluorescer chemiluminescent systems.

As outlined above, chemical light is produced by mixing an oxalate ester and hydrogen peroxide together in the presence of a catalyst and a fluorescer. Typically, fluorescers are chosen that are peroxide stable to provide a long lasting glow. In most instances, a single fluorescer has been used to produce a particularly colored light. In some cases, two or more fluorescers of essentially equivalent stability in peroxide have been mixed to produce a blended color. As an example, a blue emitting fluorescer will be mixed with a red emitting fluorescer to make a pink light.

Of the numerous fluorescers outlined above, relatively few emit light in peroxyoxalate chemiluminescence and are sufficiently peroxide stable (five phenylethynyl anthracenes, one violanthrone, and three perylene dicarboximides) to yield commercially viable products. While other fluorescers are known to emit light they are not peroxide stable, and have historically been rejected for commercial use. Other details on chemilumenisence are found in U.S. Pat. No. 6,267,914, which is incorporated herein by reference.

Another cool light source is a light emitting diode (LED). LEDs emit light when connected in a circuit. A semi-conductor chip is at the heart of an LED, which is enclosed by a clear or colored epoxy case. This chip is then connected to a circuit. LEDs operate at relative low voltages between about 1 and 4 volts, and draw currents between about 10 and 40 milliamperes. The chip has two regions separated by a junction. The p region is dominated by positive electric charges, and the n region is dominated by negative electric charges. The junction acts as a barrier to the flow of electrons between the p and the n regions. Only when sufficient voltage is applied to the semi-conductor chip, can the current flow and the electrons cross the junction into the p region. Light is generated inside the chip, a solid crystal material, when current flows across the junctions of different materials. The composition of the materials determines the wavelength and therefore the color of light. About 30 percent of the light generated inside the chip makes it way out of the brightest LEDs. Semiconductor materials have very high indices of refraction and so can trap a great deal of light when configured in a square chip.

LEPs, which are organic semiconducting materials, and LEDs, which are inorganic semiconductors, generate light in similar ways. However, light from LEPs can be patterned like liquid crystal displays. LEPs are also thin and can be flexible.

The PPV polymer or derivatives form the active layer of most promising LEP devices. Varying the chemical composition of the PPV polymer changes its physical and electro-optical properties.

Some LEP devices can be as bright as a cathode ray tube (around 100 candelas per square meter), with luminous efficacies between 2 to 3 lumens per watt. Researchers have been able to achieve brightness as high as 3 million candelas per square meter without heat degradation by operating LEP devices in pulsed mode, according to Cambridge Display Technology. Latest LEP device results from the company show luminous efficacies of 3 lumens per watt and 21 lumens per watt for the blue and green LEPs respectively. Cambridge Display Technology further reports that in collaboration with Seiko Epson, they have been refining the material and device design to produce devices with common architectures and emission suitable for continuous spectrum color displays.

Despite being able to efficiently produce high energy light in these small packages the uses to date have been for making toys, personal emergency beacons as well as traffic lights. There has been no attempt to produce a wearable light bandage for the purpose of treating disorders of the skin or teeth.

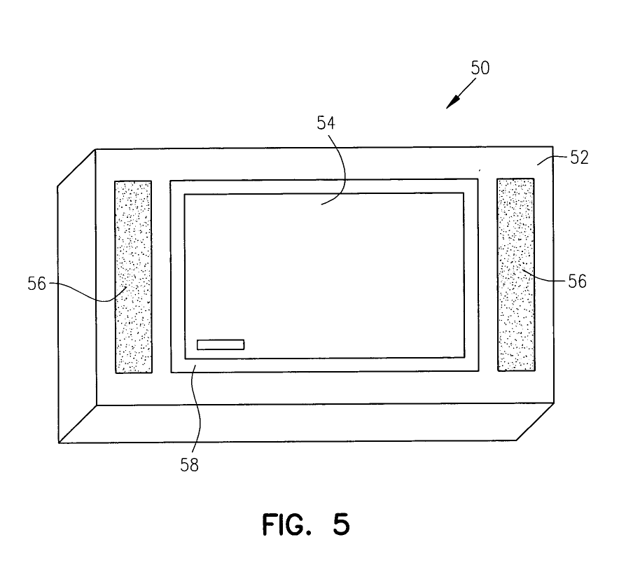

One embodiment of a light-energy-emitting device would take the form of a patch or bandage that could be affixed to or held adjacent to the area of treatment. FIG. 5 shows a light-emitting patch 50 according to one embodiment. The patch includes a flexible or moldable material 52 which can be cut to almost any size or shape. The material can be hydro-colloid, a flexible material, a moldable material, a polymer material, a flexible water repellant material, for example. A light emitting surface 54 as described above is incorporated into a surface of the patch material. Adhesive surfaces 56 can be applied on one or more areas of the patch to allow the patch to be temporarily applied to a surface. A reflecting surface 58 can be configured around or behind a portion of the light-emitting surface to reflect light back to the treatment surface. Reflecting surface 58 can be a foil surface or mirrored surface.

FIG. 13 shows a light-emitting device 180 according to one embodiment. Device 180 includes a patch as discussed above mounted upon a tissue 182. Device 180 includes a transparent or partially transparent front surface and a light-emitting material or member (not shown) within or coupled to the device, as discussed above and below. A reflective surface 184 is incorporated into the back surface of the device. In one embodiment, reflective surface 184 can include one or more curved focusing mirrors 186. In such a use, for example, an original light is emitted towards the tissue 182 and partially reflected light goes back to the focusing reflecting mirror 186 and is focused and intensified back to the treating surface tissue 182. Some embodiments can include a reflective surface which is shaped in order to focus the reflected light back to a desired point on the surface or into the depth of the tissue.

In other embodiments, mirrors 186 can include a filtered mirror in that the light entering the mirrored surface and exiting the mirrored surface will be filtered. This will allow for a screening out of unwanted wavelengths of light being reflected back to the treating surface. This can be of importance when the absorption characteristics of the targeted chromophore is altered after the absorption of the initial light exposure and one wishes to concentrate the subsequent light exposure to a specific wavelength or set of wavelengths. An example of this is when treating a oxygenated blood vessel with a light or laser source. After targeting the vessel with an appropriate wavelength or set of wavelengths that are preferentially absorbed by oxygenated blood, such as but not limited to 532 nm or 577-600 nm, the blood is partially or wholly altered to have a a portion of deoxygenated blood in the targeted vessel. Deoxygenated blood has a different absorption curve than oxygenated blood with absorption peaks in the infrared portion of the electromagnetic spectrum. Thus, one could use a filter on or adjacent to the mirror which allows a greater proportion of light in the infrared portion, such as 1064 nm light, to be reflected back to the targeted vessel.

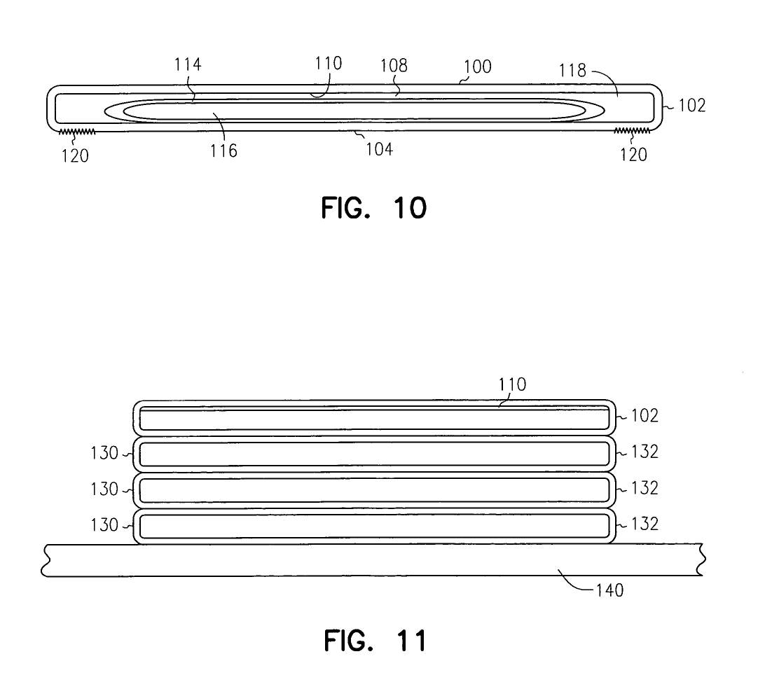

FIG. 10 shows a light-emitting device 100 according to one embodiment. Device 100 can be an enclosed or partially enclosed container 102 that has at least one side 104 being transparent or partially transparent to let out only partial or filtered light. One or more of the other sides, such as distal inside surface 108 may have a reflecting surface 110 so as to direct the light through the transparent side 104. Device 100 can be formed of the materials discussed above, for example.

Inside the container 102 there may be a sealed pouch 114 containing a dye 116 surrounded by a liquid activator 118. In some examples, the activator may be partially held in place by a clear or partially opaque “mesh” inside the container. Applying pressure to the container 102 ruptures the inner pouch 114 and the two materials 116 and 118 are mixed to start the light reaction. Side 104 can include an adhesive 120 to temporarily attach the container to tissue. The container 102 can be in any configuration in size and shape in order to conform to the particular use, as will be discussed below.

FIG. 11 shows a light-emitting device 130 according to one embodiment. Light-emitting device 130 include a container 132 that can be made out of all transparent material. In some examples, the side edges of container 132 can block light with the top and bottom surfaces being transparent. In one example use, one or more containers 132 can be placed on the treating surface 140 on top of one another several containers high. A top device 102, being most distal to the treating surface would have a reflecting surface 110 on the back so as to amplify the total light dose per area.

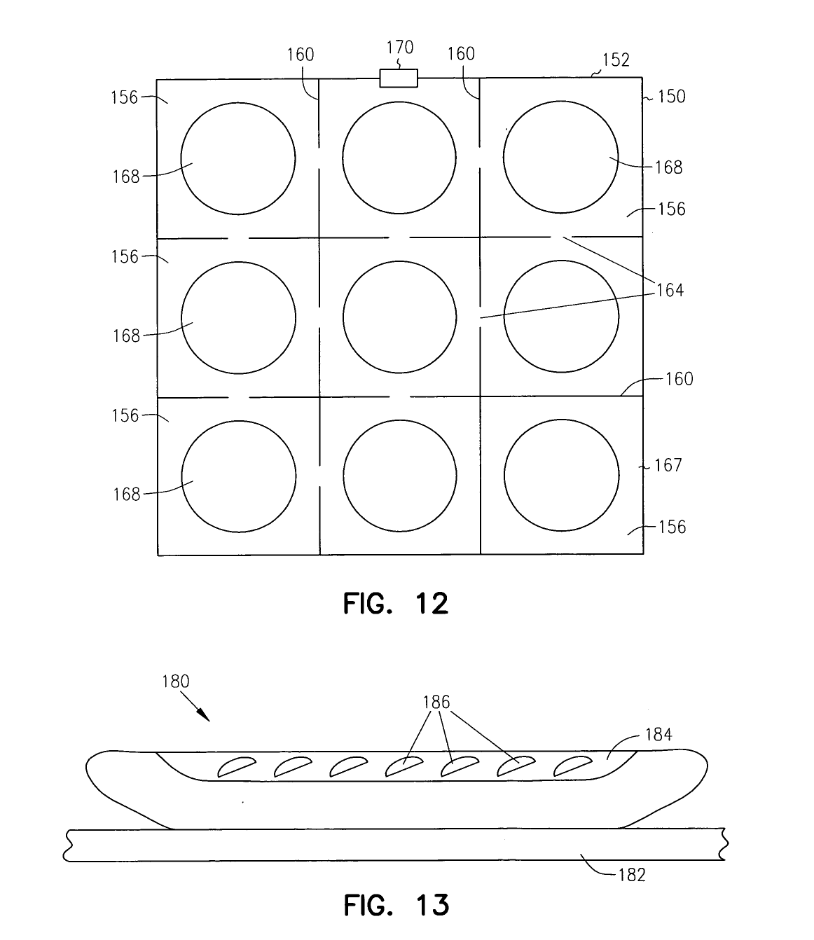

FIG. 12 shows a light-emitting device 150 according to one embodiment. Device 150 includes a container 152 having chambers 156 that are initially empty. The chambers 156 can be separated by battens, baffles, or barriers 160. In various embodiments, openings 164 can be between chambers 156 so as to allow the fluid to mix but also to keep it fairly uniform within the container. Thus, the fluid will not sink to the lowest level. In other embodiments, one or more of the chambers, such as chamber 167 can be closed off so there is no mixing.

In this embodiment, the exciter and dye will be contained in one or more sealed pouches 168 that when squeezed empty their contents into one of the chambers. The chambers 156 could be filled at the same time or at differing times and the dye and accelerators use to fill each container could be the same so as to emit the same wavelengths of light and rate of exposure of different with any combination of wavelengths and or activators. The reaction may give off some gas so there may be one or more valves 170 on each container to let the excess gas out while keeping the fluid inside. As will be discussed below, this type of container can be in the shape of a mask or any other shape so as to conform to the treating area.

Other embodiments include almost any type of light-emitting container; alternate forms could include a stick, such as a child's chemiluminescent glow-stick, or a wand of similar design.

The light-emitting container, could be affixed to or held adjacent to, the external body part using any type of adhesive, string, binder, tie or wrap.

The light-emitting container is designed to treat any external surface of a human or animal body, such as, but not limited to skin or teeth.

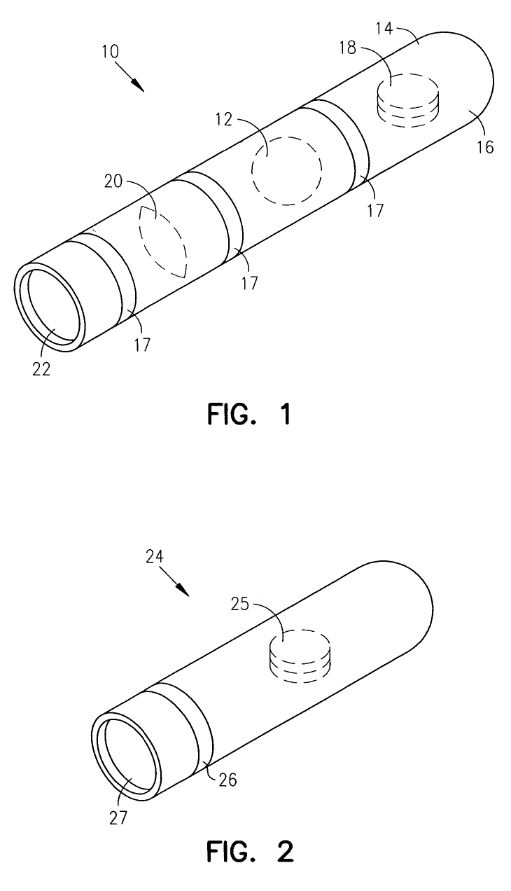

FIG. 1 shows a perspective view of a light-emitting device or container 10 according to one embodiment. In this example, the light-emitting source 12 is within a hand-held container 14 having a handle 16. This container can be used for short-term application of light energy. This example includes an optional energy source 18, such as a battery, a light emitting member 12, a lens 20, and includes a light-emitting end 22 to be placed next to treatment surface. One or more light reflecting surfaces 17 can be incorporated in the device. For example, a reflecting surface 17 can be located near the light-emitting end 22 to reflect light energy back at the treatment surface.

FIG. 2 shows a perspective view of a light-emitting container 24 according to one embodiment. This example includes a light-emitting member 27 at an end of the device and an optional energy source 25 within the device. One example includes a reflective surface 26.

### Example Uses of the Embodiments

Teeth Whitening

The past method of whitening the external surfaces of teeth includes repeated home application of a bleaching agent such as a hydrogen peroxide mixture to a custom dental mold, which is applied to the teeth nightly for several weeks. There are faster methods employed in a dentist office using a bleaching agent, which is photo-activated. The material is applied in a similar fashion as the home treatment but is then irradiated by a light source (usually blue light) in the dentist's office for 20 min per area. The total treatment can take up to two hours.

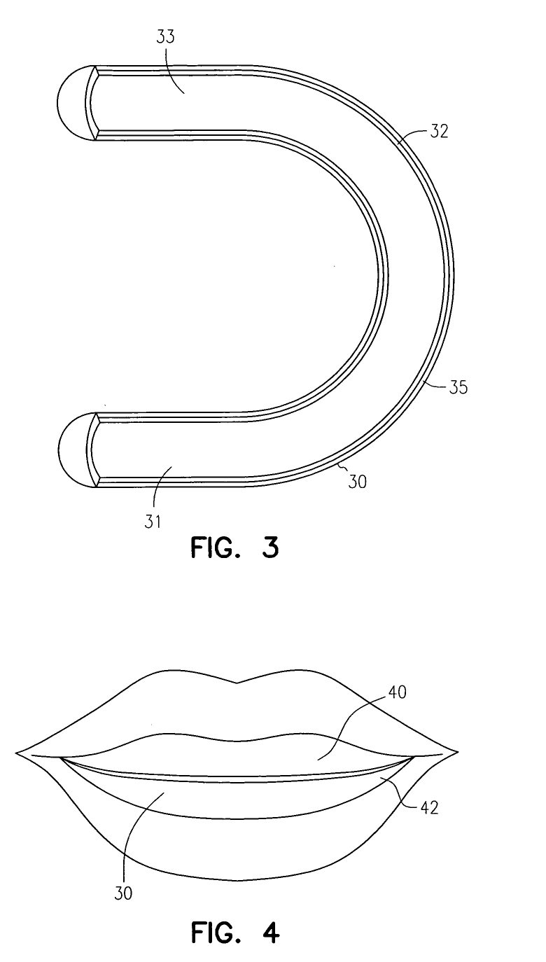

FIGS. 3 and 4 show examples of dental trays 30 and 40 incorporating light-emitting materials or features in accordance with one embodiment.

Referring to FIG. 3, a top view of dental tray 30 is shown. In one embodiment, dental tray 30 is molded to include a cavity 31 such that dental tray 30 fits over upper or lower teeth of a patient. A light emitting material 32 can be located within the custom dental tray, which can be filled with a light activated bleaching agent 33. The light activated material 32 is made to evenly irradiate the teeth with blue light for the desired amount of time. Light-emitting material 32 can be a light-emitting patch, device, or container as discussed above, which can be temporarily positioned within dental tray 30. Tray 30 can be molded and set into place at the dentist's office and the patient can then go home and remove the device at a designated time. The light-emitting surface 32 can extend throughout the dental tray or be only on selected portions of the tray. A reflective surface 35 can be provided to reflect light back to the teeth.

In some example uses, a hydrogen peroxide or a carbamide can be painted on the teeth or impregnated in a strip applied adjacent to the teeth. The light emitting tray 30 is then placed over the teeth. In another example, the hydrogen peroxide or carbamide is placed within the cavity of the tray and then the tray is applied to the teeth.

In various embodiments, the light energy used can include blue light with the addition of a material to provide a small exothermic reaction that causes the release of heat so as to increase the temperature of the H₂O₂ or carbamide ( more specifically used in percentages between 1% and 35%) to help accelerate the reaction but not too much so that the intra-pulp temperature does not raise greater than 5 degrees C. In some embodiments, other blended wavelengths can be used to optimize the treatment. Some embodiments utilize white, red, or blue light alone or in combinations of wavelengths. In some embodiments, devices having light intensities and characteristics as shown below in Table 1 of the Precancer and Cancer Treatment section can be utilized.

FIG. 4 shows an example of dental trays 30 and 40 mounted within a mouth 42.

Examples:

For several years hydrogen peroxide used alone and with a light source has been used to successfully whiten stained teeth. There are basically two methods employed. The first uses a lower percentage of hydrogen peroxide either in toothpaste, strip or as a gel with a professionally made mouth guard. These are generally very safe and effective, yet they take as long as 2 to 4 weeks in order to get the desired results. The second technique is performed in the dentist's office and uses a high concentration of hydrogen peroxide and laser or blue light. This procedure takes at least one hour and requires a professional dam to be constructed to protect the gingiva. This procedure is quite expensive due to the equipment needed as well as the space and time it takes up in the dentist's office.

The aim of this study is to compare a teeth-whitening system that employs a disposable light source to the conventional BriteSmile technique (BriteSmile Inc. Walnut Creek, Calif.).

Methods:

Study One

Routinely extracted molars were mounted in a plaster base and kept hydrated prior to and during testing. The teeth were either treated with the BriteSmile™ standard protocol or treated with the BriteSmile™ gel and illuminated with blue light patches (Table 1). The patches were changed at 20-minute intervals. The gel was rinsed off all teeth and reapplied at 20-minute intervals for a total of 60 minutes of treatment. In other examples, the light can be left in place for 20 to 60 minutes or more and a new light patch or container applied at anytime. The Table shows pre-and post-treatment results using the Lumin shade guide scale.

Study Two

Routinely extracted molars were soaked in tea and coffee and then mounted in a plaster base and kept hydrated prior to and during testing. The teeth were either treated with the BriteSmile™ standard protocol or treated with the BriteSmile™ gel and illuminated with blue red or white light patches (Table 1). The patches were changed at 20-minute intervals. The gel was rinsed off all teeth and reapplied at 20-minute intervals for a total of 60 minutes of treatment. In other examples, the light can be left in place for 20 to 60 minutes or more and a new light patch or container applied at anytime.

TABLE I
Study One

| Number | TX | Pre op | Post op |
|---|---|---|---|
| 1 | BS | A3.25 | A1.5 |
| 2 | BS | A3.5 | A2 |
| 3 | Blue LP | A3 | A2 |
| 4 | Blue LP | A3.5 | A2 |
| 7 | Blue LP | A3.5 | A2 |
| 9 | Contr. | A3.5 | A3.5 |

Study Two

| Number | Treatment | Pre | Post |
|---|---|---|---|
| 1 | BriteSmile | D4 | A2 |
| 2 | BriteSmile | D4 | A2 |
| 3 | Bleach no light | D4 | C3 |
| 4 | Red Light | D4 | C6 |
| 5 | Red Light | D4 | C6 |
| 6 | White Light | D4 | D6 |
| 7 | White Light | D4 | D6 |
| 8 | Blue Light | D4 | B4 |
| 9 | Blue Light | D4 | B4 |

In other embodiments, light energy of approximately 9.5 J/cm² or less can be delivered to the teeth over a period of approximately 90 minutes or less. Some embodiments deliver energy of approximately 7.0 J/cm² or less over a period of approximately 90 minutes or less. Some embodiments deliver light energy of approximately 5.0 J/cm² or less over a period of approximately 90 minutes or less. Some embodiments deliver light energy of approximately 3.0 J/cm² or less over a period of approximately 90 minutes or less. Some embodiments deliver light energy of approximately 2.0 J/cm² or less over a period of approximately 90 minutes or less. In some embodiments, the light-emitting devices can give an exposure of approximately 0.67 to approximately 1.8 J/cm² over a period of 40 to 90 minutes respectively. In some embodiments, the exposure can be approximately 65 mJ/cm² to approximately 100 mJ/cm over a period of 90 minutes. In other embodiments, the optimum energy and rate of delivery using these low dose light sources can be varied. In some examples, new light patches are reapplied when needed.

Acne Treatment

Studies have shown that the bacteria that causes acne is susceptible to blue light exposure. There are currently several blue light sources that are used to treat acne. The treatment involves shining a blue light at the treatment area for 20 to 30 minutes while in the physician's office.

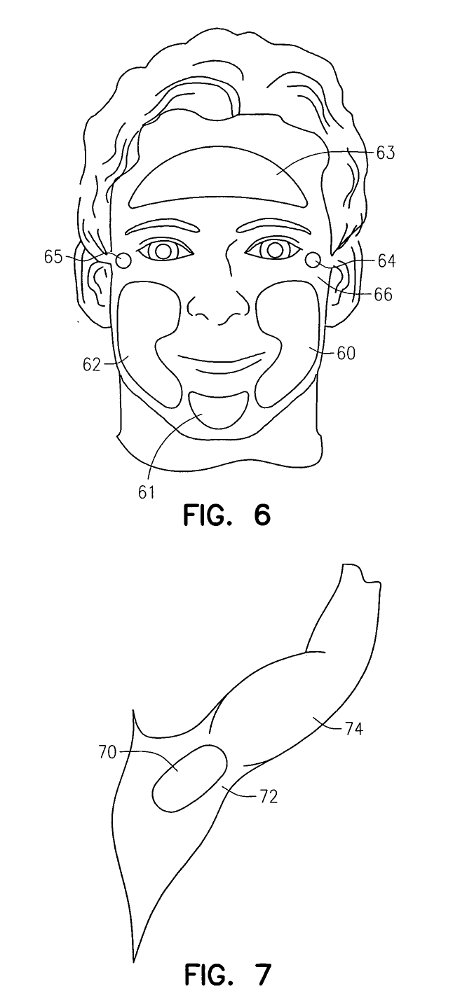

FIG. 6 shows an example application of light-emitting devices, such as patches 60, 61, 62, 63, 64, and 65, to a face 66 for treatment of acne. In one embodiment, light emitting patches 60-66 are adherent to or held adjacent to the skin. The patches can be made to emit blue light for a designated time and energy. For example having the energy and intensity of the light emitting devices described above for teeth whitening. The patches can be made or cut to any size and affixed to the affected area at night in order to give the desired light dose to treat the lesion. Some embodiments use a red light to decrease the inflammatory component of the acne. Other blended wavelength can be used to optimize the treatment. For example, some embodiments use light to match the fluorescence of the corporoporphrins in P acne that would be blue, red and or yellow alone or in combination. In some example uses, the patches can be applied for a duration of 20 minutes to 90 minutes, 2 times per day to 2 times per week. Topical agents can be incorporated into the patch, such as benzoyl peroxide, salicylic acid, flagyl, erythromycin, clindamycin, etc.

In one embodiment a light-emitting face mask can be utilized to deliver the light for treatment.

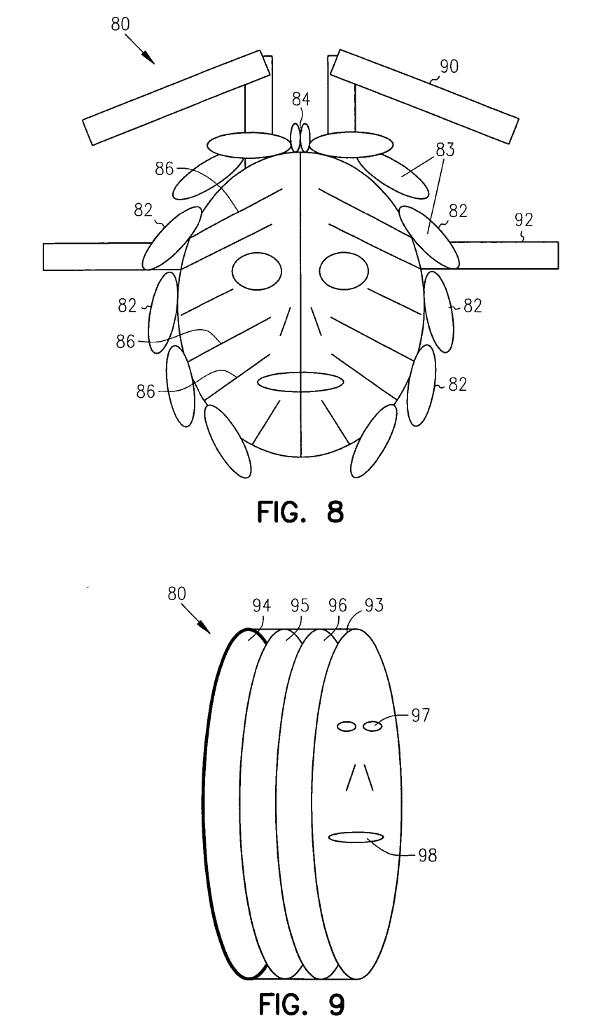

FIGS. 8 and 9 show a light-emitting mask 80 according to one embodiment.

In one embodiment, face mask 80 has a plurality of cavities 82 in which ampoules 83 of dye and exciters can be introduced at various times. There can be one or more escape valves 84 to allow gas to exit, since the chemical reaction will create a bit of gas. In one example, one or more baffles 86 can be placed in a certain direction so that the liquid dye can mix well and also will not pool in the bottom of the mask. In use, the mask can be placed over the face with straps 90 and 92 used to hold the mask on. A combination of ampoules 83 can be introduced into the face mask using one or more of cavities 82.

FIG. 9 shows schematically a perspective side view of mask 80. In one embodiment, mask 80 can include separate layered chambers 94, 95, 96 to allow different or the same fluids in the separate chambers. A front inner surface 93 can include a reflective surface. The mask includes opening 97 and 98 for eyes and mouth.

Wart Treatment

Human papilloma virus is susceptible to high dose visible light. Many warts are resistant to multiple treatment modalities.

In one embodiment, the current invention describes a light-emitting device, such as a light-emitting patch that can emit yellow and or green light to be affixed to the wart for a desired length of time to destroy the wart tissue. In some embodiments, an exothermic patch can be provided and used with a blue, red, or infrared light. For example, a heat-generating layer can be incorporated into the patch, as known. The heat-generating layer can include a mixture of oxidizable materials (e.g., oxidizable metal powder(s)) and carbon or activated carbon powder. Examples of oxidizable metal powders include, are but not limited to, iron, aluminum, magnesium, zinc, and a mixture thereof. Other oxidizing material that can be used in the present invention to generate heat include (e.g., ferrosoferric oxide, plumboblumbic oxide, trimanganese tetroxide, black copper oxide and manganese dioxide in the form of fine particle). The heat-generating layer can also contain electrolytes/salts. The electrolytes/salts include, but are not limited to the salts of sodium, potassium, lithium, calcium, iron, magnesium, and aluminum. Examples of electrolytes include, but are not limited to, NaCl, KCl, LiCl, CaCl.sub.2, FeCl.sub.3, FeCl.sub.2, MgCl.sub.2, AlCl.sub.3, Na.sub.2 SO.sub.4, K.sub.2 SO.sub.4, Fe (SO.sub.4) .sub.3, FeSO.sub.4, or MgSO.sub.4.

In some example uses, the patches can be applied for a duration of 20 minutes to 90 minutes, 2 times per day to 2 times per week. Some examples apply the patch for 90 minutes or longer. Some embodiments utilize patches having the energy and intensity as discussed above in the teeth whitening section.

Precancer and Cancer Treatment

Certain pre cancers and cancers are susceptible to light based treatments such as actinic keratoses, basal cell and squamous cell cancer. Several modes of light energy have been employed to treat these conditions such as X-radiation, water absorbing infrared radiation as well as visible light in combination with a photosensitizing agent such as topical aminolevulinic acid (ALA).

This embodiment describes a light based patch that could be configured to produce visible or infra-red light in order to be used alone or in combination with a photosensitizer to treat skin cancers. Currently, there is FDA approval for the use of topical ALA and visable light for treating pre-cancerous lesions such as actinic keratoses. The ALA is applied to the treatment area 24-48 hours prior to irradiation with visible light. The device described in this embodiment could be constructed to deliver the correct amount of light energy and wavelength to activate the photosensitizer by applying it to the treatment area for an appropriate amount of time after the application of the ALA.

This patch with the use of a photosensitizer such as ALA, photophrin, rose Bengal, lyme, begomot, celery oil, or other photosensitizer could also be used to treat other conditions such as removal of unwanted hair, warts and psoriasis.

Example

Six subjects with 47 actinic keratosis were selected for treatment. Photographs were obtained prior to treatment. The treatment area was swabbed with 20% ALA and occluded for 45-90 minutes. A light patch was then applied for 40-90 minutes. Instructions were given to protect the area from light for 72 hours. Patients were assessed for clearance of lesions, post-op pain and side effects at day 1, 7 and 14 and 3 or 6 months.

Results: Patients had no sensation at time of treatment. Two to twenty-four hours post treatment patients felt the sensation of sunburn with associated erythema and superficial erosions. The erosions healed within 2 weeks. Preliminary data shows clearance of 68% of the lesions at last follow up.

Conclusion: This pilot study demonstrates complete clearance of actinic keratosis at 2 months follow up after PDT treatment with short contact ALA and a novel light patch.

Actinic keratosis are premalignant lesions of the skin caused by excessive sun exposure. They appear as rough scaly white, yellow, brown or red patches on sun exposed skin. More than fifteen percent of the US population live with these lesions. These lesions may degenerate into squamous cell carcinomas (SCC) at a rate noted to be between 0.1% -20%. In the US the risk of sun induced SCC's to metastesize is approximately 4% with 2% being fatal. Because of these figures most advise treating these precancerous lesions when they occur.

This pilot study evaluates a photodynamic therapy employing short contact ALA and novel low energy light source for the treatment of actinic keratosis.

Evaluation of the Light Patches:

The spectral shift vs. time was measured for each patch. The patch was placed in a light-tight box with a fiber optic placed directly over the patch and leading to an spectrometer (Ocean Optics S2000, Ocean Optics). The patch was activated and readings were collected at 10 minute intervals for 10 hours. The readings were corrected by subtracting a dark level reading taken prior to the activation of the patch.

The intensity was then measured over time by using both a direct and indirect method. For the direct method the activated light patch was placed in front of a chopper thirteen centimeters from the photodetector (1 cm² area). Power readings were collected at 10 minute intervals for 4 hours. A dark level was collected prior to the activation of each patch. A total of three patches for each color were tested and their values were averaged. The area of the detector was 1 cm² and it was fully illuminated.

The power was also measured in an indirect method of “pseudo-power” by multiplying the maximum number of counts for each spectra by its full width at half maximum (FWHM) to simulate the area under each curve.

Clinical Study:

Subjects with at least one clinically diagnosed actinic keratosis on the face were selected from a clinical dermatology practice. After informed consent each lesion was swabbed with alcohol and then swabbed with 20% ALA (Kerastick, DUSA Pharm. Wilmington, Mass.). The area was then occluded. After 45 to 60 minutes the occlusive dressing was removed and the area was covered with a chemilumenscent light patch, as described herein, for 40 to 90 minutes. The patches emitted either blue light or white light. The subjects were told to stay out of the sun and follow up in 1, 4 and 12 or 24 weeks. Photographs were taken prior to treatment and at each follow-up visit. Clinical examination was also performed by pre-treatment and at each follow up visit. The actinic keratosis were documented as either completely resolved no resolution.

### Results

The spectral outputs were measured by Spectra Med (Providence, R.I.) ( Table 1). Six patients (5 male and 1 female) average age of 71 years (54-81) were enrolled with a total of 47 AK's (Table 2). The subjects experienced mild stinging or burning occurring after the treatment and throughout the first 24 hours after treatment. This discomfort was mild and did not require any pain control. In all patients there were superficial erythema, crusting and/or erosions that occurred during the first 48 hours after treatment lasting for 1-2 weeks. There were no significant side effects or scarring from the treatment. There were no statistically significant differences noted between the clinical response and the duration of ALA incubation color of light used or exposure time. The erosions healed within 2 weeks. Thirty-two of the forty-seven lesions(68%) were cleared at last follow up which was from 11 to 28 weeks (15.8 weeks average).

TABLE 1

Color

| | Blue | White |
|---|---|---|
| Spectral Peak (nm) | 455, 490 | 445, 545 |
| Initial Patch Radiance | 1.1830 | 1.4196 |
| (mW/cm² r) | | |
| Emitted Energy @ | 33 | 53 |
| 20 min.(mJ/cm²) | | |
| Emitted Energy @ | 71 | 88 |
| 25 min.(mJ/cm²) | | |
| Emitted Energy @ 40 min.(J/cm²) | 1.06 | 0.67 |
| Emitted Energy @ 45 min.(J/cm²) | 1.15 | 0.7 |
| Emitted Energy @ 90 min.(J/cm²) | 1.8 | 0.9 |

TABLE 2

| | | | | | | | | Reduction |
|---|---|---|---|---|---|---|---|---|
| | | | Color of | ALA exp | Light exp | AK Pre- | AK Post- | (%)/Follow |
| Pt # | Sex | Location | Light | (Min.) | (Min.) | Tx | Tx | (Wks) |
| 1 | M | R | Blue | 45 | 45 | 6 | 3 | 50/11 |
| | | forehead | | | | | | |
| | | L | Blue | 45 | 45 | 5 | 2 | 60/11 |
| | | forehead | | | | | | |
| 2 | M | R cheek | White | 60 | 90 | 3 | 1 | 66/15 |
| | | L cheek | Blue | 60 | 90 | 4 | 2 | 50/15 |
| 3 | F | R cheek | White | 45 | 40 | 4 | 2 | 50/11 |
| | | L cheek | Blue | 45 | 40 | 3 | 1 | 66/11 |
| 4 | M | R | Blue | 45 | 45 | 5 | 0 | 100/28 |
| | | temple | | | | | | |
| | | L temple | Blue | 45 | 45 | 3 | 0 | 100/28 |
| 5 | M | L temple | Blue | 45 | 45 | 9 | 2 | 78/12 |
| 6 | M | L cheek | Blue | 45 | 45 | 5 | 2 | 60/16 |
| Total | | | | | | 47 | 15 | 68/(15.8) |

Discussion:

Photodynamic therapy with aminolevulinic acid (ALA), and blue light is FDA cleared for the treatment of actinic keratosis. Topical ALA is preferentially taken up by precancerous cells and converted to a fluorescent molecule protoporphyrin IX (PpIX) via the hemoglobin biosynthesis pathway. Protoporphyrin IX fluoresces through an oxygen dependant mechanism when activated by specific wavelengths of light. This activation produces singlet oxygen, which can further react to form superoxide and hydroxyl radicals to kill the surrounding cells.

There are several studies showing the effects of ALA PDT in clearing actinic keratosis using different ALA incubation times and light parameters. The FDA cleared system involves applying a 20% ALA solution and allowing 14 to 18 hours before illumination. The area is then exposed to a blue light source (BLU-U Blue Light Photodynamic Therapy Illuminator, DUSA Pharm, Wilmington Mass.) which is designed to give a 10 J/cm² light dose at 417±5 nm, over 16 minutes and 40 seconds. Clinical studies using this system showed an average of about 66% of subjects obtaining complete clearing at 8 weeks follow-up. Despite the good results from this treatment the therapy is inconvenient requiring two visits for treatment and the therapy can be quite painful.

This pilot study showed actinic keratosis can be cleared using a short ALA incubation time and low rate of light exposure with minimal healing time and no significant discomfort or adverse effects. This study used an occluded 45-60 minute incubation of ALA instead of 11 to 12 hours. Although, this longer incubation time has been shown to give peak fluorescence other studies show considerable PpIX concentration in skin 2-4 hours after topical ALA administration.

The accumulation of PpIX during the short incubation time in this study may have been enhanced by the fact that the areas were occluded. Although, there appears to have been a clinically relevant amount of PpIX fluorescence with the parameters used in this study. The amount could be enhanced with longer incubation times, which may lead to greater clearance of these lesions. This study did not demonstrate that longer incubation times (40 vs 60 minutes) had a significant different effect. This may be due to the low number of subjects evaluated.

Despite this decreased energy a clinical reaction and response was seen using lower doses. The light patches used in this study gave an exposure of 73 to 92 mJ/cm² over a period of 40 to 90 minutes respectively. This is considerably less than the 10 J/cm² given over a period of 16 minutes and 40 seconds delivered by the Dusa's Photodynamic Therapy System. It could be due to the fact that the light was delivered over longer exposure times avoiding the possibility of photobleaching. It has been shown that there is fluorescent activity of PpIX at energies as low as 3 mW/cm2 when exposed to red light, since ALA has a 5-10 fold greater absorption of blue light one would expect that there would be activation at 0.3 mW/cm2. Further studies are underway in order to further delineate the optimum energy and rate of delivery using these low dose light sources in order to be able to shorten the light exposure time as well.

In some examples, light energy of approximately 9.5 J/cm² or less can be delivered over a period of approximately 90 minutes or less. Some embodiments deliver energy of approximately 7.0 J/cm² or less over a period of approximately 90 minutes or less. Some embodiments deliver light energy of approximately 5.0 J/cm² or less over a period of approximately 90 minutes or less. Some embodiments deliver light energy of approximately 3.0 J/cm or less over a period of approximately 90 minutes or less. Some embodiments deliver light energy of approximately 2.0 J/cm² or less over a period of approximately 90 minutes or less. In some embodiments, the light patches can give an exposure of approximately 0.67 to approximately 1.8 J/cm² over a period of 40 to 90 minutes respectively. In some embodiments, the exposure can be approximately 65 mJ/cm to approximately 100 mJ/cm² over a period of 90 minutes. In other embodiments, the optimum energy and rate of delivery using these low dose light sources can be varied. In some examples, new light patches are reapplied every 20-25 minutes. One embodiment includes first abrading the stratum corneum off to enhance the penetration of the topical sensitizer.

Anti-Photoaging

Photoaging is characterized histologically by elastotic changes in the dermis, thinning of the epidermis, irregular pigmentation and increased ectatic blood vessels. Currently there are several laser and high energy light based devices to treat these symptoms by targeting water to remove the epidermis and superficial dermis or by selectively targeting the unwanted vasculature, pigment and non-ablatively injuring the dermis in order to create a wounding response that can shift the metabolic balance of the skin to create new healthy epidermis and dermis.

In one embodiment, patches or facemasks as discussed above can be used for treatment. In one embodiment, a light based patch can be constructed to create a single wavelength or a plurality of wavelengths that can target these structures to effect an improvement in the photoaged skin. For example, some embodiments use blue, red, infrared, and/or yellow alone or in combination. Some embodiments include growth factors such as epidermal growth factors and keratinocyte growth factors in the patch. Some embodiments can include anti-oxidants such as vitamin C, nitroxides, or superoxides incorporated into the patch. In some example uses, the patch or facemask can be applied for a duration of 20 minutes to 90 minutes, 2 times per day to 2 times per week. Some examples apply the patch for 90 minutes or more. Some embodiments include energy and light intensity characteristics as discussed above in the Teeth Whitening section and in the Precancer and Cancer Treatment section.

Light Assisted Hair Removal

Laser and light sources have been used for several years in order to permanently remove unwanted hair. This is done primarily by using wavelengths that get absorbed by melanin in the hair shaft and follicle. By targeting this pigment the light absorption creates heat which by reaching a threshold temperature can destroy the hair shaft. By limiting the pulse duration and creating a cooling effect on the skin during the treatment one can spare the more superficial structures which contain pigment such as the epidermis while selectively injuring the deeper larger hair shaft and follicle. For example, different activators, as discussed above, could be added to the patch that allow the light energy to be delivered within a given time frame.

The patches described in this embodiment can be constructed to produce light visible and or infrared in a specified pulse duration and energy in order selectively injures hair follicles. Some embodiments use blue, red, infrared, and/or yellow alone or in combination.

In one example, a patch would be applied to the treatment area for a specified time in order to effect the response needed. Employing heating (using an exothermic patch as discussed above, for example) to heat the tissue thus reducing the amount of light needed to effect the desired response and/or cooling to protect the surface structures could be employed to enhance the results. Also, combining a photosensitizer or hair growth retardant, such as elforatine, in the patch could enhance the results. One example incorporates liposomal melanin into the patch. FIG. 7 shows a light emitting patch 70 applied to the axillae 72 under arm 74 for removing unwanted hair according to one embodiment. In some example uses, the patch can be applied for a duration of 20 minutes to 90 minutes, 2 times per day to 2 times per week. Some examples apply the patch for 90 minutes or more. Some embodiments include energy and light intensity characteristics as discussed above in the Teeth Whitening section and in the Precancer and Cancer Treatment section.

Treatment of Inflammatory Skin Disorders

Many inflammatory skin disorders such as psoriasis and eczema can be successfully treated with light based therapy. One of the problems with this type of therapy is that it is difficult to target only the diseased tissue without exposing the non-diseased tissue to the light based treatment.

In one embodiment, a patch as discussed above can be used for treatment. Such a patch can be constructed to produce ultraviolet light or visible light, such as blue, red and or yellow alone or in combination. The patch can treat these inflammatory disorders as well as be able to be cut to fit the disordered plaques and left on for the desired time to get the optimum effect. The patches can also be made to have active topical medications that can enhance the treatment outcome, such as cortisone, elidel, and protopic, for example. Some embodiments include an anti-metabolite for psoriasis or hyperproliferative disorder, such as 5-flourouracil. In some example uses, the patches can be applied for a duration of 20 minutes to 90 minutes, 2 times per day to 2 times per week. Some examples apply the device for 90 minutes or longer. Some embodiments include energy and light intensity characteristics as discussed above in the Teeth Whitening section and in the Precancer and Cancer Treatment section.

Wound Sterilization

Many bacteria, fungi and viruses (such as herpes simplex) are susceptible to light based energy.

In one embodiment, a light-emitting device such as a patch as discussed above can be used for treatment. Such a patch can be constructed to produce ultraviolet light or visible light, such as blue, red and or yellow alone or in combination. The patches and bandages described in this embodiment can be constructed to produce light energy either alone or in combination with electrical thermal energy or topical agents (such as bacitracin, bactroban, etc.) in order to optimize the ability to kill specific organisms. The patch can then be applied to the infected area be that an ulceration, erosion or wound of any kind or an infected appendage such as a finger or toenail infected with a fungus. Anti-fungals can be incorporated into the patch such as nystatin, lotriman, clotrimazole, etc. The patch or bandage can be left on for various times and reapplied as needed in order to effect an optimum response. In some example uses, the patches can be applied for a duration of 20 minutes to 90 minutes, 2 times per day to 2 times per week. Some examples apply the device for 90 minutes or more. Some embodiments include energy and light intensity characteristics as discussed above in the Teeth Whitening section and in the Precancer and Cancer Treatment section.

Ulcers and Sores

Another example of an external wound requiring treatment, would be a decubitus ulcer, pressure ulcer, or bed sore. These wounds are complicated by, and have delayed healing due to infection by micro-organisms. The infection in such wounds is susceptible to light-energy, and can be treated by applying a light-emitting patch or bandage on, over or adjacent to the affected area.

In one embodiment, a patch as discussed above can be used for treatment. Such a patch can be constructed to produce ultraviolet light or visible light, such as blue, red and or yellow alone or in combination. In some example uses, the patches can be applied for a duration of 20 minutes to 90 minutes, 2 times per day to 2 times per week. Some examples apply the device for 90 minutes or more. Some embodiments include energy and light intensity characteristics as discussed above in the Teeth Whitening section and in the Precancer and Cancer Treatment section.

Treatment of Scars and Tattoos

Scars and tattoos on the skin, are able to be treated with light energy. The patches and bandages described in this embodiment can be constructed to produce light energy that when applied on, over, or adjacent to such lesions cause beneficial effects. In one embodiment, a patch as discussed above can be used for treatment. Such a patch can be constructed to produce ultraviolet light or visible light, such as blue, red and or yellow alone or in combination. In some example uses, the patches can be applied for a duration of 20 minutes to 90 minutes, 2 times per day to 2 times per week. Some examples apply the device for 90 minutes or more. Some embodiments include energy and light intensity characteristics as discussed above in the Teeth Whitening section and in the Precancer and Cancer Treatment section.

Other Examples

In one or more embodiments, naturally occurring oils that are photosensitizing can be used with one or more of the light emitting devices discussed above. Some examples are lemon, orange, mandirine, ammi visnaga, angelica archangelica seeds, melissa officinalis, cinnamomum cassia, cinnamomum verum leaves and bark. Other naturally occurring citrus oils can be used as well. The naturally occurring photosensitizers can be used with any example discussed above, such as acne, AK, anti-photodamage, hair removal, and psoriasis.

In various embodiments, the present system provides a concept of incorporating therapeutic light energy in a container, such as a patch or bandage or facemask, that can be affixed to, or held adjacent to or over, external surfaces of a human or animal body, for the treatment of body sites such as skin or teeth. The light can be specified as having certain wavelengths, energy pulse durations, and directed specifically to the area needing treatment. The light-containers can be constructed to contain active topical agents, drug delivery mechanisms, and have the ability to elaborate electrical and thermal energy to enhance the therapeutic effects. For example, the patches or devices can employ materials of different polarity in order to create an electrical current. In other examples, electrodes can be incorporated into the device and internal or external energy can be supplied to deliver an electrical current to the tissue.

In one example, the present system can include a process for producing a container that emits light energy, that adheres to or can be positioned adjacent to an external surface of the body. The system includes a patch or bandage shape member and a light source of specific wavelength, intensity and duration of exposure. In various embodiments, the light source can include a light source comprised of a cool light device, a light source comprised of a chemiluminescent material, a light source comprised of an electroluminescent material, a light source composed of a light emitting diode, and a light source comprised of a light-emitting polymer. The light source can be totally self contained or have an external power supply. The patch or bandage can be adapted to be affixed, applied to, or positioned adjacent to, the treatment area of skin or teeth, and can include a hydro colloid dressing, a flexible adherent material, a moldable polymer material, or a flexible water repellant material.

One aspect includes a bandage or patch that has a reflecting surface so as to direct the light and reflected light to the treatment surface. One aspect includes a bandage or patch that contains other topical preparations to enhance the effect of the light therapy. One aspect includes a bandage or patch that contains a topical delivery system for driving topical preparations into the treatment area.

The bandage or patch can include an ability to produce an electrical field that can effect the treatment area by driving product into the treatment area or creating electrical or thermal energy that can enhance the therapeutic effect of the light energy. The bandage or patch can include the ability to create a thermal reaction such as a hyper- or hypothermal reaction to enhance the therapeutic effect of the light energy.

One aspect provides a dentifrice that can be custom molded in a specific configuration so as to be used as a dental tray for teeth whitening or cleaning.

One aspect provides a patch or bandage that can be applied either by adhesives, straps, ties or binders of any type.

As used herein, wavelengths are: Red light 780-622 nm; Orange light 622-597 nm; Yellow light 597-577 nm; Green light 577-492 nm; Blue light 492-455 nm; Violet light 455-390 nm.

It is understood that the above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled.

1. An apparatus comprising:
a device having a first surface adapted to be placed against a tissue; and
a light emitting member attached to the patch and configured to deliver light energy of approximately 9.5 J/cm² or less through the first surface over a period of approximately 90 minutes or less.

2. The apparatus of claim 1, wherein the device includes an adhesive on the first surface of the device.

3. The apparatus of claim 1, wherein the device includes a flexible structure to substantially conform to a shape of the tissue.

4. The apparatus of claim 1, wherein the light emitting member includes a chemiluminescence source.

5. The apparatus of claim 1, wherein the device includes a reflective surface to reflect light towards the first surface.

6. A method comprising:
applying a photosensitizing agent to a tissue; and
irradiating the photosensitizing agent with a light having an energy of approximately 9.5 J/cm² or less.

7. The method of claim 5, wherein irradiating includes applying a chemiluminescence patch to the tissue.

8. The method of claim 5, wherein the photosensitizing agent is applied and the tissue is occluded for approximately 90 minutes or less before irradiating the tissue.

9. The method of claim 5, wherein irradiating includes irradiating for approximately 90 minutes or less.

10. A method comprising:
applying a photosensitizing agent to an area of tissue;
occluding the area for approximately 90 minutes or less; and
irradiating the area with a light for approximately 90 minutes or less.

11. The method of claim 10, wherein the area is occluded for approximately 45 minutes to approximately 90 minutes.

12. The method of claim 10, wherein the area is irradiated for approximately 40 minutes to 90 minutes.

13. An apparatus comprising:
a dental tray having a cavity shaped to fit over teeth; and
a light emitting material within the cavity and located so as to emit light on the teeth when the dental tray is applied over the teeth.

14. The apparatus of claim 13, further comprising a reflective surface proximate the light emitting material.

15. The apparatus of claim 13, wherein the light emitting material is located on preselected portions of the dental tray.

16. The apparatus of claim 13, wherein the light emitting material includes a chemiluminescent light source.

17. The apparatus of claim 13, wherein the light emitting material emits a blue light.

18. The apparatus of claim 13, wherein the light emitting material emits a red light.

19. The apparatus of claim 13, wherein the light emitting material emits a white light.

20. The apparatus of claim 13, wherein the dental tray is adapted to hold a hydrogen peroxide material.

21. An apparatus comprising:
a dental tray for holding a hydrogen peroxide material, the dental tray adapted to be applied over teeth; and
a light source located within the dental tray and positioned to deliver illumination to the hydrogen peroxide material.

22. The apparatus of claim 21, wherein the light source is a temporary light source having an active life of approximately 20 minutes.

23. The apparatus of claim 21, wherein the light source delivers a blue light.

24. The apparatus of claim 21, wherein the light source delivers a red light.

25. The apparatus of claim 21, wherein the light source delivers a white light.

26. The apparatus of claim 21, wherein the light source includes a chemiluminescent patch adapted to be temporarily affixed to the dental tray.

27. A method comprising:
applying a hydrogen peroxide to one or more teeth;
placing a dental tray over the teeth; and
illuminating the teeth with a light source located within the dental tray.

28. The method of claim 27, wherein the hydrogen peroxide is a hydrogen peroxide gel.

29. The method of claim 27, wherein the teeth are illuminated for approximately 20 minutes to approximately 60 minutes.

30. The method of claim 27, wherein the light source is a blue light.

31. The method of claim 27, further including heating the hydrogen peroxide with an exothermic reaction from the light source.

32. An apparatus comprising:
a device having first surface which is at least partially transparent and is adapted to be to be placed on a tissue;
a chemiluminescent light source within the device to emit light through the at least partially transparent surface; and
one or more baffles within the device to retard a flow of the chemiluminescent light source.

33. The apparatus of claim 32, wherein the device includes a valve.

34. The apparatus of claim 32, wherein the light source emits a blue light.

35. The apparatus of claim 32, wherein the light source emits a red light.

36. The apparatus of claim 32, wherein the light source emits a yellow light.

37. An apparatus comprising:
a facemask having a first surface which is at least partially transparent and a second surface and at least one chamber between the first surface and the second surface; and
a chemiluminescent light source located within the chamber.

38. The apparatus of claim 37, wherein the facemask includes a plurality of baffles to retard the flow of the chemiluminescent light source.

39. The apparatus of claim 37, wherein the facemask includes a valve.

40. The apparatus of claim 37, wherein the second surface includes a reflective surface.

41. A method of treating acne comprising:
applying a light emitting container directly to a face having acne; and
transmitting light from the light emitting container to the acne until a desired light dose has been delivered.

42. The method of claim 41, wherein the light emitting container is a patch containing a chemiluminescent light source.

43. The method of claim 41, wherein the light emitting container is a facemask containing a chemiluminescent light source.

44. The method of claim 41, wherein the light emitting container emits a blue light.

45. The method of claim 41, wherein the light emitting container emits a red light.

46. The method of claim 41, wherein the light emitting container emits a yellow light.

47. The method of claim 41, wherein the light emitting container is applied for approximately 20 minutes to approximately 90 minutes.

48. A method of performing an antiphotoaging treatment, the method comprising:
applying a light emitting container directly to an area of skin having photoaging symptoms; and
transmitting light from the light emitting container to the area until a desired light dose has been delivered.

49. The method of claim 48, wherein the light emitting container is a patch containing a chemiluminescent light source.

50. The method of claim 48, wherein the light emitting container is a facemask containing a chemiluminescent light source.

51. The method of claim 48, wherein the light emitting container emits a blue light.

52. The method of claim 48, wherein the light emitting container emits a red light.

53. The method of claim 48, wherein the light emitting container emits a yellow light.

54. The method of claim 48, wherein the light emitting container is applied for approximately 20 minutes to approximately 90 minutes.

55. A method of treating warts, the method comprising:
applying a light emitting container directly to an area having a wart; and
transmitting light from the light emitting container to the area until a desired light dose has been delivered.

56. The method of claim 55, wherein the light emitting container is a patch containing a chemiluminescent light source.

57. The method of claim 55, wherein the light emitting container emits a blue light.

58. The method of claim 55, wherein the light emitting container emits a red light.

59. The method of claim 55, wherein the light emitting container emits a yellow light.

60. The method of claim 55, wherein the light emitting container emits a green light.

61. The method of claim 55, wherein the light emitting container further emits heat from an exothermic reaction within the light emitting container.

62. The method of claim 55, wherein the light emitting container is applied for approximately 20 minutes to approximately 90 minutes.

63. A method to remove hair, the method comprising:
applying a light emitting container directly to an area targeted for hair removal; and
transmitting light from the light emitting container to the area until a desired light dose has been delivered.

64. The method of claim 63, wherein the light emitting container emits a light in the visible spectrum.

65. The method of claim 63, wherein the light emitting container emits an infrared light.

66. The method of claim 63, wherein the light emitting container is a patch containing a chemiluminescent light source.

67. The method of claim 63, wherein the light emitting container further emits heat to the area as a result of an exothermic reaction within the light emitting container.

68. A method treating an inflammatory skin disorder, the method comprising:
applying a light emitting container directly to an area of skin having an inflammatory skin disorder; and
transmitting light from the light emitting container to the area until a desired light dose has been delivered.

69. The method of claim 68, wherein the light emitting container emits a light in the visible spectrum.

70. The method of claim 68, wherein the light emitting container emits an ultraviolet light.

71. The method of claim 68, wherein the light emitting container is a patch containing a chemiluminescent light source.

72. The method of claim 71, wherein the patch is shaped to cover the area of skin having the inflammatory skin disorder without exposing non-diseased tissue to the light.

73. A method of sterilizing a wound, the method comprising:
applying a light emitting container to a wounded area; and
transmitting light from the light emitting container to the area until a desired light dose has been delivered.

74. The method of claim 73, wherein the light emitting container emits a light in the visible spectrum.

75. The method of claim 73, wherein the light emitting container emits an ultraviolet light.

76. The method of claim 73, wherein the light emitting container is a patch containing a chemiluminescent light source.

77. The method of claim 73, wherein the light emitting container further includes a topical agent to kill bacteria.

78. A method of treating a scar or tattoo, the method comprising:
applying a light emitting container to an area having a scar or tattoo; and
transmitting light from the light emitting container to the area until a desired light dose has been delivered.

79. The method of claim 78, wherein the light emitting container emits a light in the visible spectrum.

80. The method of claim 78, wherein the light emitting container is a patch containing a chemiluminescent light source.

81. A method comprising:
applying a naturally occurring, photosensitizing oil to an area of skin; and
applying a light emitting container to the area; and
transmitting light from the light emitting container to the area until a desired light dose has been delivered.

82. The method of claim 81, wherein the naturally occurring, photosensitizing oil includes a citrus oil.

83. The method of claim 81, wherein the light emitting container includes a patch containing a chemiluminescent light source.
